(19) United States
(12) Patent Application Publication
(10) Pub. No.: US 2002/0165577 A1
(43) Pub. Date: Nov. 7, 2002

(54) Easily detachable ultrasonic clamping device

(75) Inventors: David Alan Witt, Mainsville, OH (US); Craig N. Faller, Milford, OH (US); Jeffrey David Messerly, Cincinnati, OH (US); Chester O. Baxter,III, Loveland, OH (US); Richard F. Schwemberger, Cincinnati, OH (US); Steven K. Neuenfeldt, Cincinnati, OH (US); Kevin Lee Houser, Springborok, OH (US); Chester G. Haibel,Jr, Mason, OH (US)

Correspondence Address: Leopold Presser, Esq., Scully, Scott, Murphy & Presser, 400 Garden City Plaza, Garden City, NY, 11530, US

(73) Assignee: Ethicon Endo-Surgery, Inc., Cincinnati, OH

(21) Appl. No.: 09/849,905

(22) Filed: May 4, 2001

(51) Int. Cl. A61B 17/28

(52) U.S. Cl. 606/205

(57) ABSTRACT

An ultrasonic surgical instrument which incorporates a thumb and finger ring-actuated structure for the ultrasonic clamping device which is finger-operated in a scissor-like mode. Provided is either a fixed or removable blade tip and clamp assembly for ultrasonic treatments, so as to impart to the surgical instrument the ability of providing a variety of essentially exchangeable tips while reducing operationally engendered vibrations and stresses at the point of the coupling thereof to the clamp arm. The assembly is actuatable by relative movements between a blade extender and an outer tubular sheath extending to a handle end connected to the thumb and finger ring-actuated structure.

### BACKGROUND OF THE INVENTION

1. Field of the Invention

The present invention relates to an ultrasonic surgical device which incorporates a readily detachable ultrasonic clamping device. In particular, the invention is directed to the provision of a removable blade tip and clamp assembly for ultrasonic treatments, so as to impart to the surgical device the ability of providing a variety of essentially exchangeable tips while reducing operationally engendered vibrations and stresses at the point of the coupling thereof to the clamp arm.

Ultrasonic surgical devices or instruments which include ultrasonically-operating shears require the employment of a clamping mechanism which clamps tissue between an ultrasonic blade and a clamp arm. The mechanism for actuating the clamping mechanism necessitates the installation of a movable inner tube which is adapted to activate the clamp arm, and a stationary tube on which the clamp arm pivots. These tubes considerably increase the expense of the surgical instrument the cost of which is passed on to the customer or medical practitioner, and ultimately to the patient. In contrast, the inventive surgical instrument is designed to eliminate unnecessary costly components while being capable of reducing the outer diameter of the instrument or device. There is also a need in the medical technology for an ultrasonic instrument which possesses a detachable tip structure in order to facilitate cleaning, disposal, or usage of various end effectors; such as the blade and clamp element. A problem is also encountered with current detachable tip instruments in that they have an excessive number of parts, rendering the tips cumbersome to construct, expensive to manufacture; and also difficult to assemble by various possibly semi-skilled personnel and medical practitioners.

Although the medical technology is extensively concerned with the problems encountered in connection with the construction of ultrasonic clamping devices or surgical instruments of generally the type considered herein, various desirable constructive and functional aspects are clearly lacking in the current state-of-the-technology.

2. Discussion of the Prior Art

Terumasa Japanese unexamined patent application 9-38099 discloses an ultrasonic surgical instrument with a tip/clamp assembly located at the distal node of a blade extender. The clamp arm of the instrument is movable on an axial pin, whereas the tip end is detachable through the use of a threaded screw connection, whereby this connection facilitates the interchanging of the tips. Due to the housing being located at the node, the point of attachment for the interchangeable tips is not located at the node, whereby the clamp assembly is not connected directly to the waveguide, but rather is attached to a housing located at the distal node of the blade extender.

Mitsumasa Japanese unexamined patent application 8-275952 discloses an ultrasonic surgical tool with a blade/clamp assembly which is affixed to the blade via a nodally mounted block. The clamp is held open by a resilient flexible material; whereas the outer tube may then be actuated over the clamp through the intermediary of a manually-operated assembly, such as a thumb and finger ring assembly for effectively closing the clamp which becomes ensheathed by the outer tube. The tip/clamp assembly may also be removed for cleaning/disposal by means of a threaded connection.

Schad U.S. Pat. No. 5,676,678 is directed to the provision of a surgical instrument with a holder for various tips, wherein the holder is coupled to an inner tube, and with the holder being detachable from the inner tube of the surgical instrument. The coupling functions by using two snap legs which engage over the tip assembly to fix the holder; the snap legs possessing elastic qualities which facilitate the selective attachment and removal of the tip assembly. When the holder coupling is exposed, the outer tube is held in place against the pressure of a helical spring by engaging hook projections located in an undercut rim of the instrument. These projections may then be released to cover and protect the coupling.

Schad German Patent 19 534 618 issued on Mar. 20, 1997 discloses a surgical instrument with jaw components whereby at least one jaw part is connected to an inner tube so that it can be actuated relative to an outer tube to which the jaws are pivotally mounted. The jaw part consists of a catch which inserts into a bore hole opening, and closing the jaw part is effected by rotating the latter around a transversely mounted pin connecting the jaw parts to the outer tube. The instrument is designed to be disassembled through the provision of a removable inner tube so as to facilitate cleaning or disposal thereof. This patented surgical instrument is designed for manual and electrical use rather than as an ultrasonic surgical device. In contrast, the present invention possesses a clamp/blade system which is more effective in ultrasonic devices.

Hood U.S. Pat. No. 5,669,922 discloses an ultrasonic surgical instrument having an end assembly comprising an end hook which is threaded and screwed into complementary threads formed on or in an extender. The blade of the instrument has a step that is located at or proximate to the nodal location of the assembly. This patent discloses only a blade, whereas to the contrary, the present invention incorporates a clamp mechanism, and moreover locates the coupling of the clamp mechanism at the node. By locating the coupling of the clamp at the node, mechanism pursuant to the present invention, vibrations and consequently stresses acting on the coupling are reduced. The nodal placement of the coupling functions to decrease wear on the coupling and reduces the possibility of clamp arm/transverse mounted pin/blade movement due to ultrasonic vibrations.

Davidson et al. U.S. Pat. No. 5,322,055 discloses an ultrasonic surgical apparatus comprising a housing, an ultrasonic element carried by the housing for generating ultrasonic vibration, a blade coupled to the ultrasonic element and a clamp which moves in opposition to the blade for use in clamped coagulation. The present invention patentably distinguishes thereover by including a removable tip area which is located in an anti-nodal region, a multitude of choices for the tube/lever closure of the clamping device, a disassembly capability and a construction possessing fewer parts.

Finally, DiMatteo, et al U.S. Pat. No. 5,810,859 discloses an ultrasonic surgical instrument with a removable waveguide which is connected to the hub of an ultrasonic transducer handle. The hub is designed to enable a coupling member to apply a rotational torque to the hub of the outer sheath, which is to be transmitted to the waveguide in order to tighten it onto the mounting device of the handpiece assembly. In a preferred embodiment of the patent, the location of the connection of the waveguide and handpiece is at the node to reduce any wear and stress due to the ultrasonic vibrations. In contrast, the present invention features a nodal connection at the point of attachment of the movable end effector; whereas although the patent discloses an embodiment with a removable end effector, that embodiment does not provide any disclosure about the nodal location of the movable end effector (jaw) coupling. The nodal location of the movable end effector pursuant to the present invention, in contrast, reduces wear on the coupling and reduces the risk of the tip attachment loosening or becoming damaged and the blade becoming damaged due to ultrasonic vibrations.

### SUMMARY OF THE INVENTION

Accordingly, in order to overcome the drawbacks and limitations encountered in the prior art, the present invention discloses an ultrasonic surgical instrument featuring an easily detachable ultrasonic clamping device incorporating a thumb/finger ring assembly actuating a removable or fixed blade assembly within an outer tube. The instrument features a clamp arm located at the distal node of the instrument, which is actuated by means of a direct drive system. The invention also discloses the structure for a possible detachable blade assembly which is located at the anti-node.

Among the advantages of the present invention there is included a thumb/finger ring assembly for controlling the amount of force applied to the instrument. A spring embodied in the limiter assembly absorbs excessive force which may be possibly applied by the physician so as to prevent potential breakage of the instrument. The present invention also features an actuating blade assembly which actuates within an outer tube. This system eliminates the need for an inner tube, while at the same time eliminating the inadvertent friction caused by instruments in the prior art actuated by outer tubes which rub on the trocar. This particular friction which is encountered in the prior art, reduces the “feel” by the surgeon or medical practitioner during activation of the instrument. Moreover, the elimination of an extra outer tube reduces the overall size or diameter of the instrument, so as to resultingly improve upon visualization. The detachable blade/tip/clamp assembly allows for easy cleaning, disposal, and quickly interchangeable tips. Though these are several embodiments of the latter, the present invention may also have these parts affixed to each other, thereby reducing the extra parts necessary to make an instrument capable of being disassembled. These fixed instruments are beneficial for use in surgical or medical procedures where an inexpensive, disposable instrument would be optimal in order to reduce healthcare expenditures. The location of the clamp arm at the node reduces vibration and stress due to ultrasonic vibrations, thereby reducing wear and the potential of any breakage of the clamp arm. The blade tip apparatus is located at or near the anti-node to transfer as much ultrasonic power as possible to the blade. A direct drive system connecting the clamp arm to the thumb/finger ring assembly of the instrument serves to provide the physician with controlled forceful blunt dissection. In effect, the novel ultrasonic surgical instrument offers a physician a system adapted to apply a more controlled force in both opening and closing of the clamp arm.

Pursuant to a specific embodiments, the present invention uses a pin passing through the node and some method of grounding the clamp to the tube, in the case where the blade is moving relative to the tube. In the case where the tube is moving relative to the blade, the blade/clamp interface is ground and there is force transmission from the tube to the clamp. Opening and closing could be facilitated through either blade movement relative to the tube or tube movement relative to the blade. Both movements would be initiated by the user via a manually-operated assembly, such as a thumb/finger ring assembly. The thumb and finger ring-actuated control system of the present invention provides the surgeon, physician or other medical practitioner, such as a nurse with greater control and reliability. The present invention also incorporates a direct drive system as opposed to the spring system of Mitsumasa Japanese '952. One embodiment of the present invention provides a screw-in connection that consists of a single contained piece (such as the blade tip and clamp) that may be easily removed or attached. The screw-in connection provides for faster interchange or removal of tips and reduces overall part costs. However, other attachment methods may be utilized such as by means of magnaforming, press fit, swaging, and so forth; which methods are specifically directed to the provision of non-removable tips.

Accordingly it is an object of the present invention to provide an improved ultrasonic surgical instrument which incorporates an easily detachable and replaceable ultrasonic clamping device.

Another object of the invention resides in the provision of an ultrasonic surgical instrument possessing a unique attaching structure for the clamping blade and tip device which reduces stress and vibrations encountered due to ultrasonic vibrations so as to extend the service life of the instrument.

Yet another object of the invention is to provide an ultrasonic instrument of the type described which employs a minimum number of easily assembled parts so as to render the instruments inexpensive to manufacture and render components of the instrument economically disposable and/or replaceable after only a single use.

Still another object resides in the provision of an ultrasonic instrument of the type described which incorporates features rendering the instrument replaceable and/or disposable at a low cost after only a single use by the surgeon or medical practitioner.

### BRIEF DESCRIPTION OF THE DRAWINGS

Reference may now be made to the following detailed description of preferred embodiments of ultrasonic surgical instruments pursuant to the invention, taken in conjunction with the accompanying drawings; in which:

FIG. 1 illustrates a longitudinal view, partly in section, of a first embodiment of the ultrasonic surgical instrument pursuant to the invention;

FIG. 2 illustrates, in a view similar to that of FIG. 1, a second embodiment of the inventive ultrasonic surgical instrument;

FIG. 3 illustrates a detailed view of the blade extender and blade/clamp assembly of the instrument of FIG. 2;

FIG. 4 illustrates a detailed view of an arrangement for attaching the blade/clamp tip portion of the instrument to a blade extender and tube;

FIG. 5 illustrates, on an enlarged scale, the clamp/blade tip portion of FIG. 4;

FIG. 6 illustrates a further embodiment of the actuator handle portion of an ultrasonic surgical instrument; and

FIG. 7 illustrates a diagrammatic spring limiter arrangement for minimizing stress on the clamp/blade tip assembly of the instrument of FIG. 6.

### DETAILED DESCRIPTION OF PREFERRED EMBODIMENTS

Referring in detail to the drawings, FIG. 1 is a longitudinal sectional view of an ultrasonic surgical instrument 10 constructed pursuant to one embodiment. As shown, the ultrasonic surgical instrument 10 includes a coagulating accessory clamp arm 12. Ultrasonic surgical instrument 10 comprises a handle-forming housing structure 14 including scissor-like operable thumb/finger ring assembly portion 16, 18 actuatable by the thumb and finger or multiple fingers of a user along arrow A. A blade extender 20 in the form of an elongated rod or shaft extends from the housing structure to an ultrasonic blade 21 which in this embodiment is integral therewith. A removable transducer (not shown) is located in the housing structure 14 and mounted removably to the proximal end of the blade extender 20, and is preferably a piezeoceramic transducer for converting an electrical signal, for example, a 55,500 Hz sinusoidal waveform, into a mechanical longitudinal vibration. Handle structure 14 connects to an outer sheath or tube 22 coaxially covering the blade extender 20, with the tube 22 having an end 24 fixedly attached to the housing structure, with blade extender 20 being axially slideable relative to tube 22. In the embodiment of the ultrasonic surgical instrument 10 of FIG. 1, the thumb/finger ring assembly portion 18 is adapted to rotate around a pivot boss 28 on handle 14, and is connected to the blade extender or shaft 20 by means of an insertion arm 30.

The clamp arm 12 connects directly in a pivoted manner to the ultrasonic blade 21 through the intermediary of a pin 32 about which the arm 12 may rotate. Though a pin 32 is used in this embodiment, there are numerous other means of hingedly or pivotably connecting the clamp arm 12 to the ultrasonic blade 21, such as through a rivet, screw, or the like. The clamp arm 12, which is attached to the ultrasonic blade 21, then protrudes through a hole 34 formed in the tube 22.

During operation, wherein the thumb/finger ring assembly portion 18 of the handle is pulled towards thumb/finger ring portion 16 of handle part 14 in a finger actuated scissor-motion rotating around the cam surfaces of the pivot boss 28, whereby the insertion arm 30 which is connected to the blade extender 20 either mechanically or by some mechanical transfer device (not shown), actuates the blade extender 20 distally so as to move axially within tube 22. By actuating the blade extender 20 distally, the end of the clamp arm 12 which protrudes from a hole 34 is pulled backward against the wall of tube 22. As the proximal end of clamp arm 12 is pushed forward, the clamp arm 12 rotates around pin 32, rotating clamp arm 12 toward blade 21. When the thumb/finger ring assembly portion 18 is pulled away from the handle portion 16, the insertion arm 30 pivots around the cam surfaces of the pivot boss 28, thereby actuating the blade extender 20 proximally. As the blade extender 20 moves proximally, the proximal end of the clamp arm 12 which protrudes through the hole 34 is pushed forward by the tube 22. In doing so, the clamp arm 12 rotates around pin 32 rotating the distal end of the arm away from the blade.

It is also possible to provide a reverse of this actuation with regard to the way in which the clamp arm 12 opens and closes, depending upon actuating direction in the thumb/finger ring assembly of the handle mechanism. The ultrasonic surgical instrument 10 may be built with the thumb/finger ring assembly portion 16 as the stationary actuator or with thumb/finger assembly portion 18 as the actuator mechanism, or reversely.

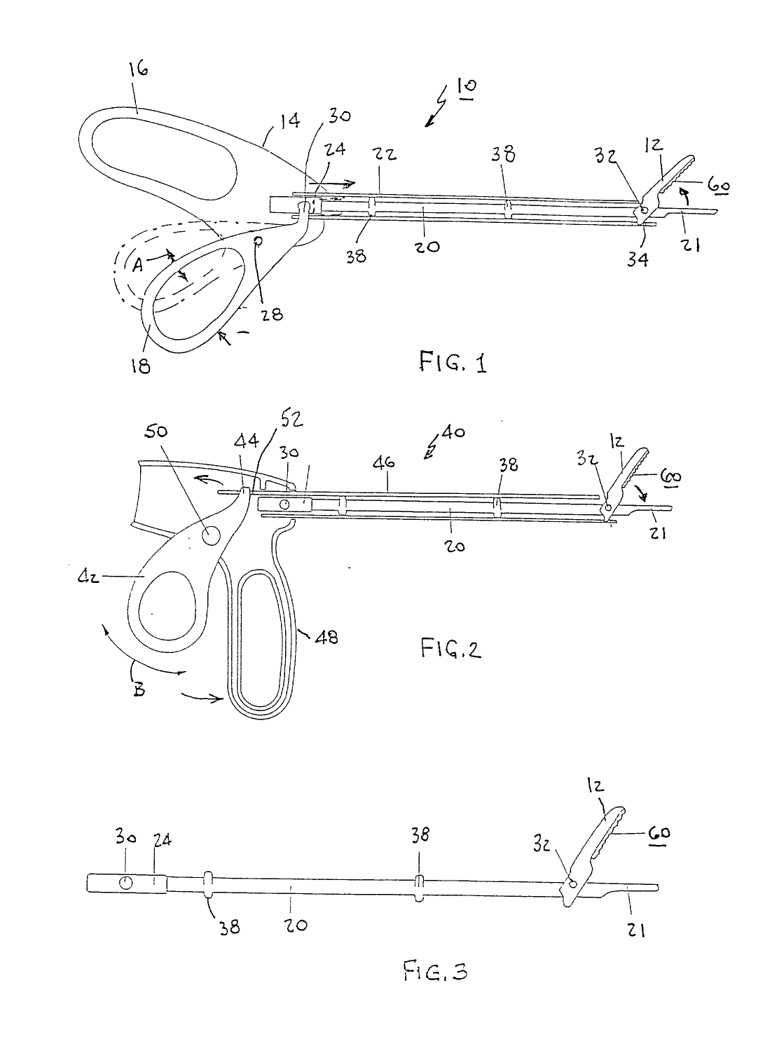

In FIG. 2, in which elements similar to those in FIG. 1 are identified by the same reference numerals, there is illustrated a second embodiment in which a thumb/finger ring assembly 42 of the instrument 40 connects by means of an actuator attachment 44 to tube 46. The assembly 42 is connected to a handle 48 through a pivot boss 50 about which assembly 42 pivots. In operation, the thumb/finger ring assembly 42 is pushed towards handle 48 rotating in directions of arrow B about the pivot boss 50. The actuator attachment 44 is connected to the outer tube 46 by projecting into a slot 52 such that the latter pivots counterclockwise around pivot boss 50, pulling the tube 46 proximally. As the tube 46 moves at its further or distal end, clamp arm 12 is pulled towards blade 21 as it pivots around pin 32. When the assembly 42 is pulled away from handle 48, the actuator attachment 44 pivots distally around the pivot boss 50, whereupon as the actuator attachment 44 pivots, and the outer tube 46 is also pushed distally. As the tube moves distally, it resultingly pushes against the proximal end of the clamp arm 12 protruding though a small hole 34. This produces the effect of rotating the clamp arm 12 around the pin 32 and pushing the clamp arm 12 away from the blade 21.

As shown in FIG. 3, rings 38 are formed from a silastic material and spaced along the length of the blade extender 20 and ultrasonic blade 21 at the nodes thereof so as to prevent the dispersion of ultrasonic waves to the tube 22.

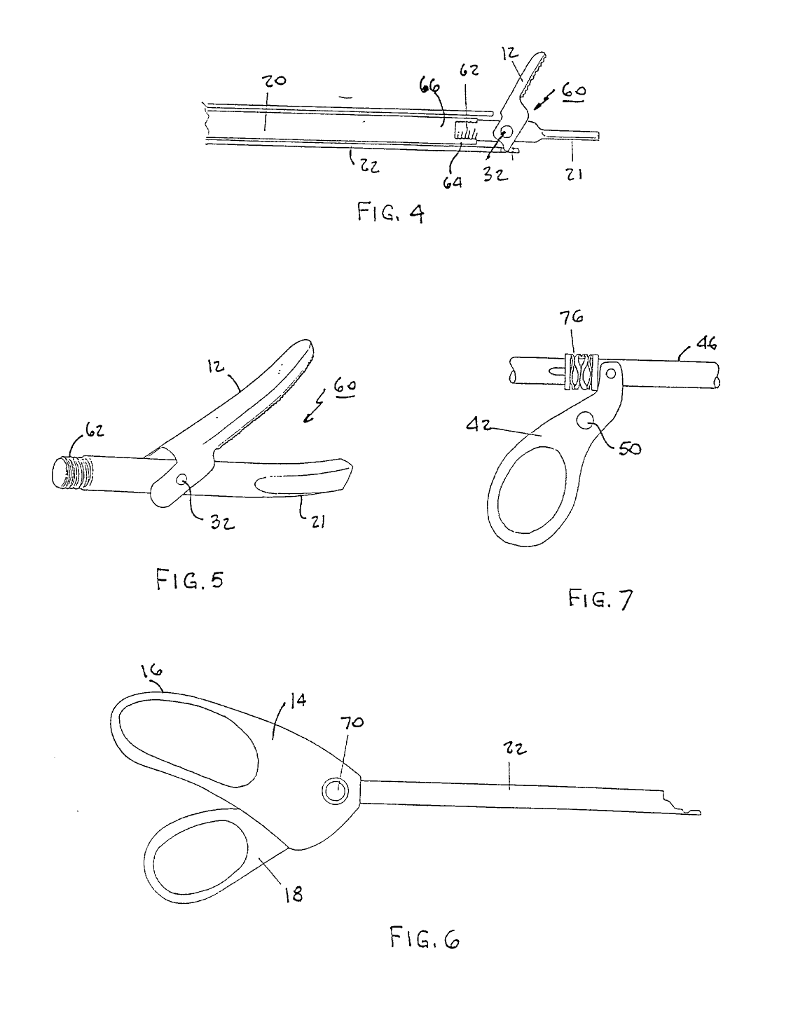

As illustrated in the embodiment of FIG. 4, the tip portion 60 of the ultrasonic surgical instrument 10 or 40, of either FIGS. 1 and 2, may be formed as a separate element from the blade extender or shaft 20 rather than being integral therewith. In this instance, the tip portion 60, which comprises the blade 21 pivotally connected by pin 32 to the clamp arm 12, as shown in detail in FIG. 5, has the blade 21 equipped with a screw threaded end 62, which is adapted to threadingly engage a complimentary screwthread 64 formed in the end 66 of the blade extender 20. This construction allows for easy removal and exchange of the tip portion 60, without requiring the entire instrument to be replaced. Other types of connections, in which the tip portion 60 is non-removable, can also be provided, such as by pin locks, interference fit, magnaforming or the like.

Referring to the schematic representation of FIG. 6, this shows the provision of a latching pushbutton 70 on the handle structure 14 of the ultrasonic surgical instrument 10 of FIG. 1, which is connected to the blade and clamp (FIG. 3), which allows for the release of the disposable blade and clamp subassembly. Depressing the pushbutton 70, which may be spring-loaded, releases the blade and clamp subassembly for easy cleaning or replacement.

In FIG. 7, a spring limiter system 76 which is located on the tube 46 acts to minimize stress transmitted to the blade assembly 60 by absorbing any excessive force applied by the physician to thumb/finger ring assembly portions 16 or 18. The spring limiter system 76 is located near the proximal end of the ultrasonic surgical instrument 40 adjacent to the handle structure 48.

While the invention has been particularly shown and described with respect to preferred embodiments thereof, it will be understood by those skilled in the art that the foregoing and other changes in form and details may be made therein without departing from the spirit and scope of the invention.

What is claimed is:

1. An ultrasonic surgical instrument comprising:
an end effector including blade and clamp means for the engagement of tissues located therebetween;
an elongated shaft element having said end effector arranged at a first end thereof;
an elongated tubular member extending about said elongated shaft element in coaxial relationship, said clamp means being movable relative to said blade and said tubular member, said elongated tubular member having a first end in operative engagement with said end effector;
a handle portion for receiving second opposite ends of respectively said elongated shaft element and said elongated tubular member, said handle portion including finger-actuatable scissors-like thumb and finger ring structure for imparting axial displacement between said elongated shaft element and said elongated tubular member, said tubular member biasing said clamp means so as to cause said blade and clamp means to selective open and close relative to each other.

2. An ultrasonic surgical instrument as claimed in claim 1, wherein said elongated tubular member is fixedly attached to said handle portion, said thumb and finger ring structure having a pivotable portion hingedly connected to the second end of said elongated shaft element, whereby actuation of said pivotable portion imparts said axial displacement to said elongated shaft element relative to said elongated tubular member.

3. An ultrasonic surgical instrument as claimed in claim 2, wherein said pivotable portion is rotatable about a pivot boss fixed to a stationary part of said handle portion.

4. An ultrasonic surgical instrument as claimed in claim 3, wherein an insertion arm on said pivotable portion is operatively engaged with said elongated shaft element for imparting axial movement thereto responsive to rotation of said pivotable portion about said pivot surfaces.

5. An ultrasonic surgical instrument as claimed in claim 1, wherein said elongated tubular member has the second end thereof slidably journaled in said handle portion, said elongated shaft element being fixedly attached to said handle portion, said thumb and finger ring assembly having a pivotable portion operatively connected with said second end of said elongated tubular member, whereby actuation of said pivotable portion imparts said axial displacement to said elongated tubular member relative to said elongated shaft element.

6. An ultrasonic surgical instrument as claimed in claim 5, wherein said pivotable portion is rotatable about a pin fastened to said handle portion, said pivotable portion including an actuating extension engageable into an aperture in said elongated tubular member for imparting the axial displacement thereto responsive to rotational movement of said pivotable portion.

7. An ultrasonic surgical instrument as claimed in claim 1, wherein said first end of said elongated shaft element and of said elongated tubular member comprise cooperative camming structure for selectively opening and closing said blade and clamp means responsive to relative axial movement between said shaft element and tubular member.

8. An ultrasonic surgical instrument as claimed in claim 1, wherein the blade of said end effector comprises a coaxial tip on said elongated shaft element.

9. An ultrasonic surgical instrument as claimed in claim 8, wherein said blade comprises a stub shaft integrally formed at the first end of said elongated shaft element, whereby said shaft element forms an ultrasonic blade extender.

10. An ultrasonic surgical instrument as claimed in claim 8, wherein said blade comprises a stub shaft which is detachably fastened to the first end of said elongated shaft element, whereby said shaft element forms an ultrasonic blade extender.

11. An ultrasonic surgical instrument as claimed in claim 10, wherein said blade is fastened to said elongated shaft element through a screw threaded connection.

12. An ultrasonic surgical instrument as claimed in claim 1, wherein said cam means comprises a cam arm mounted on said blade for pivotal movement relative thereto.

13. An ultrasonic surgical instrument as claimed in claim 1, wherein said handle portion includes latching pushbutton means for release of said blade and clamp assembly.

14. An ultrasonic surgical instrument as claimed in claim 1, wherein a plurality of axially spaced silastic rings are formed at nodes along the length of said elongated shaft element and blade so as to prevent dispersion of ultrasonic waves to said surrounding elongated tubular member during operation of said instrument.

15. An ultrasonic surgical instrument as claimed in claim 1, wherein spring limiter means are formed on said elongated tubular member so as to absorb excessive operating forces and stresses generated responsive to actuation of said handle portion.
